(19) United States
(12) Patent Application Publication
(10) Pub. No.: US 2021/0154116 A1
(43) Pub. Date: May 27, 2021

(54) COMPOSITIONS AND METHODS FOR HAIR

(71) Applicant: L'Oreal, Paris (FR)

(72) Inventors: Heather LEE, Wayne, NJ (US); Jun LIANG, Staten Island, NY (US)

(21) Appl. No.: 17/101,206

(22) Filed: Nov. 23, 2020

Related U.S. Application Data
(60) Provisional application No. 62/941,415, filed on Nov. 27, 2019.

(51) Int. Cl. A61K 8/41; A61K 8/73; A61K 8/37; A61K 8/34; A61K 8/31; A61Q 5/12

(52) U.S. Cl. CPC A61K 8/416; A61K 8/737; A61K 8/37; A61K 2800/34; A61K 8/31; A61Q 5/12; A61K 2800/5426; A61K 8/342

(57) ABSTRACT

The instant disclosure relates to compositions and methods for treating, caring for, and/or conditioning keratin fibers, such as hair. The compositions comprise a combination of cationic and hydrocarbon compounds. The methods comprise applying the compositions to keratin fibers, such as hair.

This application claims priority to U.S. Provisional Patent Application No. 62/941,415, filed Dec. 27, 2019.

### TECHNICAL FIELD

The present disclosure relates to compositions and methods for treating, caring for, and/or conditioning keratin fibers, such as hair.

### BACKGROUND

Consumers desire natural compositions for treating, caring for, and/or conditioning keratin fibers, such as hair. Such compositions can be difficult to produce as their materials must be sourced from natural and/or organic sources such as plants, while maintaining desirable performance properties.

In addition, consumers may expect some compositions to have certain properties, such as viscosity or rheology, to be recognized as a specific product—e.g., a hair conditioner, a body moisturizer, a face moisturizer, etc. —or to be convenient for dispensing and using. It is known to use conventional thickeners in such compositions in order to give the compositions a certain viscosity or rheology. However, chemicals and raw materials used in these conventional compositions may lack sustainable sourcing and therefore not comply with “green” manufacturing processes, which may make the compositions less desirable to consumers.

The present disclosure therefore relates to compositions, e.g. cosmetic compositions, that can treat, care for, and/or condition keratinous materials, wherein the compositions have desired cosmetic and composition properties, while being comprised of materials that are of sustainable and/or natural and/or organic sourcing, and/or which are produced by green manufacturing processes. Without intending to be limiting, the compositions may impart one or more benefits such as suppleness, softness, moisturization, detangling, smoothness, discipline, and/or frizz control to keratin materials.

### SUMMARY

It has surprisingly been found that compositions according to the disclosure, which may in certain embodiments be comprised of materials that are of sustainable and/or natural and/or organic sourcing, impart conditioning properties to keratinous materials such as skin or hair, including suppleness, softness, moisturization, detangling, smoothness, discipline, frizz control, and other cosmetic benefits to keratin materials, while maintaining appropriate body, volume, fullness, movement and bounce, that is comparable to non-natural or non-organic commercial formulations.

In one embodiment, the compositions comprise (a) at least one first cationic compound chosen from esterquat compounds, (b) at least one second cationic compound chosen from (i) cationic surfactants other than esterquat compounds, or (ii) cationic polymers, (c) at least one hydrocarbon oil, (d) optionally at least one glyceryl ester, and (e) at least one fatty alcohol.

In an embodiment, the at least one first cationic compound is present in an amount of about 0.1% to about 5%, based on the total weight of the composition. In a further embodiment, the at least one first cationic compound is distearoylethyl dimonium chloride.

In an embodiment, the at least one second cationic compound is chosen from cationic polymers. In a further embodiment, the at least one cationic polymer is present in an amount ranging from about 0.05% to about 2%, based on the total weight of the composition. In a further embodiment, the at least one cationic polymer is guar hydroxypropyltrimonium chloride.

In an embodiment, the at least one hydrocarbon oil is chosen from alkanes having from 10-30 carbons. In a further embodiment, the alkane having from 10-30 carbons is present in an amount ranging from about 0.1% to about 10%, based on the total weight of the composition. In a further embodiment, the at least one alkane is a C15-C19 alkane.

In an embodiment, the at least one fatty alcohol is present in an amount ranging from about 0.5% to about 10%, based on the total weight of the composition. In a further embodiment, the at least one fatty alcohol is chosen from cetyl alcohol, stearyl alcohol, cetearyl alcohol, behenyl alcohol, lauryl alcohol, myristic or myristyl alcohol, arachidyl alcohol, lignoceryl alcohol, ceryl alcohol, montanyl alcohol, myricylic alcohol, decyl alcohol, undecyl alcohol, or mixtures thereof. In a further embodiment, the at least one fatty alcohol is cetearyl alcohol.

In an embodiment, the at least one glyceryl ester is present in an amount of about 0.1% to about 3%, based on the total weight of the composition. In a further embodiment, the at least one glyceryl ester is chosen from bis-diglyceryl polyacyladipate-2, glyceryl behenate, glyceryl caprate, glyceryl cocoate, glyceryl erucate, glyceryl hydroxystearate, glyceryl isostearate, glyceryl lanolate, glyceryl laurate, glyceryl linoleate, glyceryl myristate, glyceryl oleate, glyceryl palmitate lactate, glyceryl sesquioleate, glyceryl stearate, glyceryl stearate citrate, glyceryl stearate lactate, glyceryl dioleate, glyceryl distearate, glyceryl laurate, or mixtures thereof. In a further embodiment, the at least one glyceryl ester is chosen from glyceryl stearate, glyceryl caprylate, or mixtures thereof.

In certain embodiments, the composition further comprises a solvent. In certain embodiments, the composition comprises from about 50% to about 95% water, by weight relative to the total weight of the composition.

In certain embodiments, the weight ratio of the total amount of the first and second cationic compounds to the total amount of hydrocarbon oil ranges from about 1:10 to about 10:1, from about 1:5 to about 5:1, or from about 1:3 to about 3:1.

In certain embodiments, the weight ratio of the total amount of the first and second cationic compounds to total amount of glyceryl esters ranges from about 1:10 to about 10:1, from about 1:5 to about 5:1, or from about 1:3 to about 3:1.

The composition can be substantially free of synthetic gums, silicones, and/or synthetic polymers. In certain embodiments, at least 85% of the materials used in the composition are plant-based and/or of plant origin.

Another embodiment involves a method for treating hair, said method comprising: (1) applying to the hair a composition comprising: (a) at least one first cationic compound chosen from esterquat compounds; (b) at least one second cationic compound chosen from (i) cationic surfactants other than esterquat compounds, or (ii) cationic polymers; (c) at least one hydrocarbon oil; (d) optionally at least one glyceryl ester; and (e) at least one fatty alcohol, and (2) rinsing the hair. In certain embodiments, step (1) is conducted after treating the hair with a shampoo.

### DESCRIPTION

The disclosure relates to compositions and methods for treating, caring for, and/or conditioning keratin fibers, such as hair. The compositions comprise synergistic combinations of cationic compounds and hydrocarbon oils. The compositions also comprise synergistic combinations of cationic compounds and glyceryl esters. The methods comprise applying the compositions to the hair.

I. Compositions

The compositions according to the disclosure comprise synergistic combinations of cationic compounds and hydrocarbon oils, and synergistic combinations of cationic compounds and glyceryl esters, which separately or in combination surprisingly and unexpectedly lead to improved properties. The compositions may, for example, be hair treatment, hair care, and/or hair conditioning compositions.

Cationic Compounds

Compositions according to the disclosure comprise (a) at least one first cationic compound chosen from esterquat compounds, and (b) at least one second cationic compound chosen from (i) cationic surfactants other than esterquat compounds, and (ii) cationic polymers.

The total amount of the at least one first cationic compound chosen from esterquat compounds may vary, but typically ranges from about 0.001% to about 20%, about 0.01% to about 15%, or about 0.05% to about 10%, based on the total weight of the composition. For example, the total amount of the at least one first cationic compound chosen from esterquat compounds may range from about 0.01% to about 10%, such as from about 0.01% to about 9%, about 0.01% to about 8%, about 0.01% to about 7%, about 0.01% to about 6%, about 0.01% to about 5%, about 0.01% to about 4%, about 0.01% to about 3%, about 0.01% to about 2%, about 0.05% to about 10%, about 0.05% to about 9%, about 0.05% to about 8%, about 0.05% to about 7%, about 0.05% to about 6%, about 0.05% to about 5%, about 0.05% to about 4%, about 0.05% to about 3%, about 0.05% to about 2%, about 0.1% to about 10%, about 0.1% to about 9%, about 0.1% to about 8%, about 0.1% to about 7%, about 0.1% to about 6%, about 0.1% to about 5%, about 0.1% to about 4%, about 0.1% to about 3%, about 0.1% to about 2%, about 0.5% to about 10%, about 0.5% to about 9%, about 0.5% to about 8%, about 0.5% to about 7%, about 0.5% to about 6%, about 0.5% to about 5%, about 0.5% to about 4%, about 0.5% to about 3%, about 0.5% to about 2%, about 1% to about 10%, about 1% to about 9%, about 1% to about 8%, about 1% to about 7%, about 1% to about 6%, about 1% to about 5%, about 1% to about 4%, about 1% to about 3%, or about 1% to about 2%, including all ranges and sub-ranges there between, based on the total weight of the composition.

The total amount of the at least one second cationic compound chosen from (i) cationic surfactants other than esterquat compounds, and (ii) cationic polymers may vary, but typically ranges from about 0.001% to about 10%, such as about 0.01% to about 5% or about 0.05% to about 2%, based on the total weight of the present composition. For example, the total amount of the cationic polymer may range from about 0.01% to about 8%, about 0.01% to about 7%, about 0.01% to about 6%, about 0.01% to about 5%, about 0.01% to about 4%, about 0.01% to about 3%, about 0.01% to about 2%, about 0.01% to about 1%, 0.03% to about 8%, about 0.03% to about 7%, about 0.03% to about 6%, about 0.03% to about 5%, about 0.03% to about 4%, about 0.03% to about 3%, about 0.03% to about 2%, about 0.03% to about 1%, 0.05% to about 8%, about 0.05% to about 7%, about 0.05% to about 6%, about 0.05% to about 5%, about 0.05% to about 4%, about 0.05% to about 3%, about 0.05% to about 2%, or about 0.05% to about 1%, including all ranges and sub-ranges there between, based on the total weight of the composition.

Cationic Surfactants

In certain embodiments, the cationic surfactants are plant-based and/or organic.

The at least one first cationic compound is a cationic surfactant chosen from esterquat compounds. As used herein, the term “esterquat compound” means a compound having both at least one ester function and at least one quaternary ammonium group. Exemplary esterquat compounds useful according to the disclosure include quaternized ester salts of fatty acids with triethanolamine, quaternized ester salts of fatty acids with diethanolalkylamines, and quaternized ester salts of fatty acids with 1,2-dihydroxypropyldialkylamines, such as methyl-N-(2-hydroxyethyl)-N,N-di(tallowacyloxyethyl) ammonium compounds, bis-(palmitoyloxyethyl) hydroxyethylmethylammonium compounds, methyl-N,N-bis(stearoyloxyethyl)-N-(2-hydroxyethyl) ammonium compounds, methyl-N,N-bis(cocoyloxyethyl)-N-(2-hydroxyethyl) ammonium compounds, or N,N-dimethyl-N,N-di(tallowacyloxyethyl) ammonium compounds. By way of non-limiting example, esterquat compounds useful according to the disclosure may be chosen from distearoylethyl dimonium chloride, Dibehenoylethyl Dimonium Chloride, Dipalmitoylethyl Dimonium Chloride, Ditallowoyl PG-dimonium Chloride, Dipalmitoylethyl hydroxyethylmonium methosulfate, Distearoylethyl hydroxyethylmonium methosulfate, and mixtures, thereof.

In one exemplary embodiment, the esterquat compound is distearoylethyl dimonium chloride, commercially available under the tradename VARISOFT EQ 65 MB, from the company Evonik.

The at least one second cationic compound may optionally be chosen from cationic surfactants other than esterquat compounds. By way of non-limiting example, cationic surfactants other than esterquats useful according to the disclosure may be chosen from behentrimonium chloride; cetrimonium chloride; quatemium-22; behenylamidopropyl-2; 3-di-hydroxypropyl-dimethylammonium chloride; palmitylamidopropyltrimethylammonium chloride; oleocetyldimethyl-hydroxyethylammonium chloride; stearamidopropyldimethyl (myristyl acetate) ammonium chloride; di(C1-C2 alkyl) (C12-C22 alkyl)hydroxy(C1-C2alkyl)ammonium salt; alkyltrimethylammonium salt in which the alkyl radical comprises 12 to 24 carbon atoms; propanetallowdiammonium dichloride; behentrimonium methosulfate; chloride and methyl sulfates of diacyloxyethyl-dimethylammonium, of diacyloxyethyl-hydroxyethyl-methylammonium, of monoacyloxyethyl-dihydroxyethyl-methyl-ammonium, of triacyloxyethyl-methylammonium, or of monoacyloxyethyl-hydroxyethyl-dimethyl-ammonium; distearyldimethylammonium chloride; behentrimonium methosulfate; or mixtures thereof.

In one exemplary embodiment, the cationic surfactant is chosen from behentrimonium chloride, cetrimonium chloride, behentrimonium methosulfate, and mixtures thereof.

Cationic Polymers

The at least one second cationic compound may optionally be chosen from cationic polymers. In certain embodiments, the cationic polymers are plant-based and/or organic.

By way of non-limiting example, cationic polymers useful according to the disclosure may be chosen from gums. Non-limiting examples of gums include acacia, agar, align, alginic acid, ammonium alginate, amylopectin, calcium alginate, calcium carrageenan, carrageenan, dextrin, gelatin, gellan gum, guar gum, hydroxypropyl guar, guar hydroxypropyltrimonium chloride, hydroxypropyl guar hydroxypropyltrimonium chloride, karaya gum, locust bean gum, natto gum, potassium alginate, potassium carrageenan, propylene glycol alginate, sclerotium gum, sodium carboyxmethyl dextran, sodium carrageenan, tragacanth gum, xanthan gum, or mixtures thereof.

Further non-limiting examples of cationic polymers useful according to the disclosure include non-cellulose cationic polysaccharides, such as guar gums containing trialkylammonium cationic groups. Suitable cationic guar gum derivatives include those given the PCPC (Personal Care Products Council, formerly CTFA, designation) of guar hydroxypropyl trimonium chloride, available commercially for example as JAGUAR C13S. Other suitable materials include that known as JAGUAR C15, JAGUAR C17, and JAGUAR C16 which is a hydroxypropylated cationic guar derivative containing a low level of substituent groups as well as cationic quaternary ammonium groups. Guar hydroxypropyl trimonium chloride, may also be available commercially for example as N-HANCE CG13 from the company Ashland. Also suitable is hydroxypropyl guar hydroxypropyltrimonium chloride, commercially available as JAGUAR 162.

Exemplary useful products are sold, for example, under the trade names JAGUAR C13S, JAGUAR C1000, JAGUAR C17, JAGUAR 162, JAGUAR C145, and JAGUAR EXCEL by the company Solvay (Rhodia).

In one exemplary embodiment, the cationic polymer may be chosen from hydroxypropyl guar hydroxypropyltrimonium chloride, commercially available under the tradename of JAGUAR 162, and guar hydroxypropyltrimonium chloride, commercially available under the tradename of JAGUAR C13S, both sold by Solvay (Rhodia), or mixtures thereof.

In a further exemplary embodiment, the cationic polymer may be chosen from guar gum, hydroxypropyl guar, hydroxypropyltrimonium guar, or hydroxypropyl guar hydroxypropyltrimonium guar, salts thereof, or mixtures thereof.

In yet a further exemplary embodiment, the cationic polymer may be guar hydroxypropyltrimonium chloride or hydroxypropyl guar hydroxypropyltrimonium chloride.

Hydrocarbon Oils

The compositions described herein comprise at least one hydrocarbon oil. Useful hydrocarbon oils include animal oils, plant oils, mineral oils, or synthetic oils. In certain embodiments, the hydrocarbon oils are plant-based and/or organic.

The hydrocarbon oil may, in certain embodiments, be non-polar. In various embodiments, the at least one hydrocarbon oil is chosen from linear or branch higher alkanes, i.e. alkanes having at least 9 carbon atoms. For example, the hydrocarbon oil may be chosen from alkanes having from 10-30 carbons, such as 12-24 carbons, or 15-20 carbons. In various embodiments, the hydrocarbon oil may be chosen from C13-C15, C15-C19, C18-C21, or C21-C28 alkanes.

The total amount of the at least one hydrocarbon oil may vary, but typically ranges from about 0.001% to about 20%, such as from about 0.01% to about 15% or about 0.1% to about 10%, based on the total weight of the composition. For example, the total amount of the hydrocarbon oil may range from about 0.01% to about 10%, from about 0.01% to about 9%, from about 0.01% to about 8%, from about 0.01% to about 7%, from about 0.01% to about 6%, from about 0.01% to about 5%, from about 0.01% to about 4%, from about 0.01% to about 3%, from about 0.01% to about 2%, from about 0.01% to about 1%, from about 0.1% to about 10%, from about 0.1% to about 9%, from about 0.1% to about 8%, from about 0.1% to about 7%, from about 0.1% to about 6%, from about 0.1% to about 5%, from about 0.1% to about 4%, from about 0.1% to about 3%, from about 0.1% to about 2%, from about 0.1% to about 1%, from about 0.5% to about 10%, from about 0.5% to about 9%, from about 0.5% to about 8%, from about 0.5% to about 7%, from about 0.5% to about 6%, from about 0.5% to about 5%, from about 0.5% to about 4%, from about 0.5% to about 3%, from about 0.5% to about 2%, from about 0.5% to about 1.5%, or from about 0.5% to about 1%, including all ranges and sub-ranges there between, based on the total weight of the composition.

Glyceryl Esters (Glycerol Esters)

In certain exemplary embodiments, compositions according to the disclosure comprise at least one glyceryl (or glycerol) ester. In some embodiments, the glyceryl (or glycerol) ester is plant-based and/or organic.

In one embodiment, the glyceryl ester may be chosen from glycerol esters of fatty acids or glyceryl esters (or glycerol fatty esters), for example, glyceryl monomyristate, glyceryl monopalmitate, glyceryl monostearate, glyceryl isostearate, glyceryl monooleate, glyceryl dioleate, glyceryl distearate, glyceryl laurate, trilaurin, triarachidin, tribehenin, tricaprin, tricaprylin, caprylic/capric triglyceride, trierucin, triheptanoin, triheptylundecanoin, triisononanoin, triisopalmitin, triisostearin, trilinolein, trimyristin, trioctanoin, triolein, tripalmitin, tripalmitolein, triricinolein, tristearin, triundecanoin, or mixtures thereof.

The at least one glyceryl ester may, in various embodiments, be chosen from:
esters of an oligomeric glycerol, especially the esters of diglycerol, in particular the condensates of adipic acid and of glycerol, for which a portion of the hydroxyl groups of the glycerols have reacted with a mixture of fatty acids, such as steric acid, capric acid, stearic and isostearic acid and 12-hydroxystearic acid, such as, in particular, those sold under the trade mark SOFTISAN 649 by the company Cremer Oleo or under the trademark SP SUPERMOL B MBAL-SS-(RB) by the company Croda, such as bis-diglyceryl polyacyladipate-2,
the arachidyl propionate sold under the trade mark WAXENOL 801 by Alzo,
phytosterol esters, triglycerides of fatty acids and derivatives thereof, such as hydrogenated cocoglycerides,
noncrosslinked polyesters resulting from the poly condensation between a linear or branched C4-C50 dicarboxylic acid or polycarboxylic acid and a C2-050 diol or polyol,
aliphatic esters of an ester resulting from the esterification of an aliphatic hydroxycarboxylic acid ester with an aliphatic carboxylic acid,
or mixtures thereof.

In certain embodiments, the at least one glyceryl ester is chosen from bis-diglyceryl polyacyladipate-2, glyceryl behenate, glyceryl caprate, glyceryl cocoate, glyceryl erucate, glyceryl hydroxystearate, glyceryl isostearate, glyceryl lanolate, glyceryl laurate, glyceryl linoleate, glyceryl myristate, glyceryl oleate, glyceryl palmitate lactate, glyceryl sesquioleate, glyceryl stearate, glyceryl caprylate, glyceryl stearate citrate, glyceryl stearate lactate, glyceryl dioleate, glyceryl distearate, glyceryl laurate, or mixtures thereof.

In one exemplary embodiment, the at least one glyceryl ester is chosen from glyceryl stearate, glyceryl caprylate, or mixtures thereof.

The total amount of the at least one glyceryl ester may vary, but typically ranges from about 0.001% to about 10%, such as from about 0.01% to about 5%, or about 0.1% to about 3%, based on the total weight of the composition. For example, the total amount of the glyceryl ester may range from stearate about 0.01% to about 8%, from about 0.01% to about 7%, from about 0.01% to about 6%, from about 0.01% to about 5%, from about 0.01% to about 4%, from about 0.01% to about 3%, from about 0.01% to about 2%, from about 0.01% to about 1%, from about 0.05% to about 8%, from about 0.05% to about 7%, from about 0.05% to about 6%, from about 0.05% to about 5%, from about 0.05% to about 4%, from about 0.05% to about 3%, from about 0.05% to about 2%, from about 0.05% to about 1%, from about 0.1% to about 8%, from about 0.1% to about 7%, from about 0.1% to about 6%, from about 0.1% to about 5%, from about 0.1% to about 4%, from about 0.1% to about 3%, from about 0.1% to about 2%, from about 0.1% to about 1%, from about 0.5% to about 8%, from about 0.5% to about 7%, from about 0.5% to about 6%, from about 0.5% to about 5%, from about 0.5% to about 4%, from about 0.5% to about 3%, from about 0.5% to about 2%, or from about 0.5% to about 1.5%, including all ranges and sub-ranges there between, based on the total weight of the composition.

Fatty Alcohols

Compositions according to the disclosure comprise at least one fatty alcohol. In various embodiments, the fatty alcohol is plant-based and/or organic.

In certain exemplary embodiments, the fatty alcohol is neither oxyalkylenated (in particular neither oxyethylenated nor oxypropylenated) nor glycerolated. The fatty alcohols can be represented by the formula R—OH, wherein R denotes a saturated (alkyl) or unsaturated (alkenyl) group, linear or branched, optionally substituted with one or more hydroxyl groups, for example comprising from 8 to 40 carbon atoms, such as from 10 to 30 carbon atoms, from 12 to 24 carbon atoms, or from 14 to 22 carbon atoms.

The fatty alcohols may be liquid or solid. The solid fatty alcohols that can be used include those that are solid at ambient temperature and at atmospheric pressure (25° C., 780 mmHg), and are insoluble in water, that is to say they have a water solubility of less than 1% by weight, preferably less than 0.5% by weight, at 25° C., and 1 atm.

The solid fatty alcohols may, for example, be represented by the formula R—OH, wherein R denotes a linear alkyl group, optionally substituted with one or more hydroxyl groups, comprising from 8 to 40 carbon atoms, preferably 10 to 30 carbon atoms, more preferably 12 to 24 carbon atoms, and even more preferably 14 to 22 carbon atoms.

In certain exemplary embodiments, it is possible to comprise, alone or as a mixture: lauryl alcohol or lauryl alcohol (1-dodecanol); myristic or myristyl alcohol (1-tetradecanol); cetyl alcohol (1-hexadecanol); stearyl alcohol (1-octadecanol); arachidyl alcohol (1-eicosanol); behenyl alcohol (1-docosanol); lignoceryl alcohol (1-tetracosanol); ceryl alcohol (1-hexacosanol); montanyl alcohol (1-octacosanol); myricylic alcohol (1-triacontanol).

In certain embodiments, the solid fatty alcohol may be chosen from cetyl alcohol, stearyl alcohol, behenyl alcohol, or mixtures thereof such as cetylstearyl or cetearyl alcohol.

In certain embodiments, liquid fatty alcohols, for example those containing C10-C34, have branched carbon chains and/or have one or more, such as 1 to 3, double bonds. They are optionally branched and/or unsaturated (C═C double bond), and for example contain from 12 to 40 carbon atoms.

The liquid fatty alcohols may be represented by the formula R—OH, wherein R denotes a C12-C24 branched alkyl group or an alkenyl group (comprising at least one C12-C24 double bond C═C), R being optionally substituted by one or more hydroxy groups. Optionally, the liquid fatty alcohol is a branched saturated alcohol. Optionally, R does not contain a hydroxyl group. Non-limiting examples include oleic alcohol, linoleic alcohol, linolenic alcohol, isocetyl alcohol, isostearyl alcohol, 2-octyl-1-dodecanol, 2-butyloctanol, 2-hexyl-1-decanol, 2-decyl-1-tetradecanol, 2-tetradecyl-1-cetanol or mixtures thereof. In one exemplary embodiment, the liquid fatty alcohol may be 2-octyl-1-dodecanol.

In certain exemplary embodiments, the compositions include one or more fatty alcohols selected from decyl alcohol, undecyl alcohol, dodecyl, myristyl, cetyl alcohol, stearyl alcohol, cetearyl alcohol, isostearyl alcohol, isocetyl alcohol, behenyl alcohol, linalool, oleyl alcohol, myricyl alcohol, or mixtures thereof. In various embodiments, the fatty alcohol may be cetearyl alcohol.

The total amount of the fatty alcohol may vary, but typically ranges from about 0.001% to about 20%, such as from about 0.01% to about 15% or from about 0.1% to about 10%, based on the total weight of the composition. For example, the total amount of the fatty alcohol may range from about 0.01% to about 10%, from about 0.01% to about 9%, from about 0.01% to about 8%, from about 0.01% to about 7%, from about 0.01% to about 6%, from about 0.01% to about 5%, from about 0.01% to about 4%, from about 0.01% to about 3%, from about 0.01% to about 2%, from about 0.01% to about 1%, from about 0.1% to about 10%, from about 0.1% to about 9%, from about 0.1% to about 8%, from about 0.1% to about 7%, from about 0.1% to about 6%, from about 0.1% to about 5%, from about 0.1% to about 4%, from about 0.1% to about 3%, from about 0.1% to about 2%, from about 0.1% to about 1%, from about 0.5% to about 10%, from about 0.5% to about 9%, from about 0.5% to about 8%, from about 0.5% to about 7%, from about 0.5% to about 6%, from about 0.5% to about 5%, from about 0.5% to about 4%, from about 0.5% to about 3%, from about 0.5% to about 2%, from about 0.5% to about 1%, from about 1% to about 10%, from about 1% to about 9%, from about 1% to about 8%, from about 1% to about 7%, from about 1% to about 6%, from about 1% to about 5%, from about 1% to about 4%, about 1% to about 3%, from about 1% to about 2%, from about 2% to about 10%, from about 2% to about 9%, from about 2% to about 8%, from about 2% to about 7%, from about 2% to about 6%, from about 2% to about 5%, from about 2% to about 4%, from about 2% to about 3%, from about 3% to about 10%, from about 3% to about 9%, from about 3% to about 8%, from about 3% to about 7%, from about 3% to about 6%, from about 3% to about 5%, from about 3% to about 4%, from about 4% to about 10%, from about 4% to about 9%, from about 4% to about 8%, from about 4% to about 7%, from about 4% to about 6%, or from about 4% to about 5%, including all ranges and sub-ranges there between, based on the total weight of the composition.

Solvents

In certain exemplary embodiments, compositions according to the disclosure comprise at least one physiologically acceptable medium, that is to say a medium that is compatible with human keratin materials such as the skin (of the body, face, around the eyes or the scalp), the hair, the eyelashes, the eyebrows, bodily hair, the nails or the lips. The physiologically acceptable medium may be chosen from water or a mixture of water and at least one cosmetically acceptable solvent. Non-limiting examples of cosmetically acceptable solvents include C2-C4 lower alcohols, such as ethanol and isopropanol; polyols, especially those containing from 2 to 6 carbon atoms, for instance glycerol, propylene glycol, butylene glycol, pentylene glycol, hexylene glycol, dipropylene glycol or diethylene glycol; polyol ethers, for instance 2-butoxyethanol, propylene glycol monomethyl ether and diethylene glycol monomethyl ether or monoethyl ether; or mixtures thereof.

In one exemplary embodiment, the composition comprises a solvent or solvent mixture in an amount up to about 99%, such as ranging from about 50% to about 95% or about 60% to about 90%, by weight relative to the weight of the composition. For example, the composition may comprise water in an amount up to about 99%, such as, for example, an amount ranging from about 50 to about 95%, or from about 60% to about 90%, by weight relative to the weight of the composition.

In some embodiments, the weight ratio of the total amount of first and second cationic compounds to the total amount of hydrocarbon oil ranges from about 1:15 to about 15:1, such as from about 1:10 to about 10:1, from about 1:5 to about 5:1, or from about 1:3 to about 3:1. In further embodiments, the weight ratio of the total amount of first and second cationic compounds to the total amount of glyceryl esters ranges from about 1:15 to about 15:1, such as from about 1:10 to about 10:1, from about 1:5 to about 5:1, or from about 1:3 to about 3:1.

Additional Components

The compositions according to the disclosure may also comprise additives chosen from nacreous agents, dyes or pigments, fragrances, mineral, plant or synthetic oils, waxes, vitamins, proteins including ceramides, vitamins, UV-screening agents, free-radical scavengers, antidandruff agents, hair-loss counteractants, hair restorers, preserving agents, or mixtures thereof. A person skilled in the art will take care to select the optional additives and the amount thereof such that they do not harm the properties of the compositions of the present disclosure.

The compositions of certain embodiments may comprise stabilizers, for example sodium chloride, magnesium dichloride or magnesium sulfate.

The compositions according to the disclosure may additionally comprise cosmetic adjuvants chosen from fragrances, pigments, chelating agents, softeners, antioxidants, opacifiers, stabilizers, moisturizing agents, vitamins, bactericides, preservatives, polymers, thickening agents, or any other ingredient commonly used in cosmetics for this type of application.

In certain embodiments, these additives are generally present in an amount ranging up to about 40% by weight of active material relative to the total weight of the composition, such as up to about 30%, up to about 20%, up to about 15%, up to about 10%, up to about 5%, such as from 0.01% to 30%.

If desired, a person of skill in the art can select the additives or amounts thereof in order to maintain the natural or organic properties of the compositions.

In some embodiments, the compositions are free or substantially free of secondary gums, silicones, and/or synthetic polymers. For example, the compositions include less than about 3%, less than about 2%, less than about 1%, or less than about 0.5% of secondary gums, silicones, and/or synthetic polymers, and in some embodiments comprise no secondary gums, silicones, and/or synthetic polymers. In some embodiments, however, the compositions may comprise secondary gums, silicones, and/or synthetic polymers. Non-limiting examples of silicones include amine-functionalized silicones (e.g., amodimethicone), dimethicone, bis-aminopropyl dimethicone, trimethyl silylamodimethicone, etc.

In certain embodiments, at least 75%, such as at least 80%, at least 85%, at least 90%, or at least 95% of the disclosed materials used in the compositions are plant-based. In one exemplary embodiment, compositions according to the disclosure demonstrate desirable cosmetic properties while comprising mostly, such as at least about 95%, natural origin ingredients.

The compositions described herein may be in any suitable physical form. Suitable forms include, but are not limited to, low to moderate viscosity liquids, lotions, milks, gel creams, creams, pastes, clays, conditioners, masks, and the like. In various embodiments, the compositions may have a lamellar phase structure.

The compositions may be packaged in a variety of different containers, such as, for example, a ready-to-use container. Non-limiting examples of useful packaging include tubes, jars, caps, unit dose packages, and bottles, including squeezable tubes and bottles, bottles configured with pump dispensers, and spray bottles.

II. Methods

The methods according to the disclosure comprise treating keratinous fibers with the compositions described herein. The methods generally comprise applying any of the disclosed compositions to hair.

By way of example, the methods may include use of the compositions as a conditioner, as part of a shampoo (e.g. a conditioning shampoo), as a pre-treatment, or as a rinse-off mask treatment. In one exemplary embodiment, the composition is a wash-out conditioner.

Methods of treating hair according to the disclosure may include applying a composition according to the disclosure to hair (wet, damp, or dry hair), allowing the composition to remain on the hair for a desired amount of time, and rinsing the composition from the hair. The composition may be applied to the hair before, during, or after other hair compositions (e.g., a shampoo, a conditioner, a mask, a cream, a lotion, a gel, etc.).

The composition may be allowed to remain on the hair for a period of time, for example from about a few seconds (e.g. about 10, 20, or 30 seconds) to about 1, 2, 3, 5, 10, 20, or 30 minutes, or longer. If desired, the hair may be heated while the composition is on the hair.

It is to be understood that all definitions herein are provided for the present disclosure only.

As used herein, the terms “comprising,” “having,” and “including” (or “comprise,” “have,” and “include”) are used in their open, non-limiting sense. The phrase “consisting essentially of” limits the scope of a claim to the specified materials or steps and those that do not materially affect the basic and novel characteristics of the compositions.

The terms “a,” “an,” “the,” and “at least one” are understood to encompass the plural as well as the singular.

Thus, the term “a mixture thereof” also relates to “mixtures thereof.” Throughout the disclosure, if the term “a mixture thereof” is used, following a list of elements as shown in the following example where letters A-F represent the elements: “one or more elements selected from the group consisting of A, B, C, D, E, F, or mixtures thereof.” The term, “a mixture thereof” does not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.”

Likewise, the term “a salt thereof” also relates to “salts thereof.” Thus, where the disclosure refers to “an element selected from the group consisting of A, B, C, D, E, F, a salt thereof, or mixtures thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

The salts referred to throughout the disclosure may include salts having a counter-ion such as an alkali metal, alkaline earth metal, or ammonium counterion. This list of counterions, however, is non-limiting.

The expression “one or more” means “at least one” and thus includes individual components as well as mixtures/combinations.

All ranges and amounts given herein are intended to include subranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. The term “about” is used herein to indicate a difference of up to +/−10% from the stated number, such as +/−9%, +/−8%, +/−7%, +/−6%, +/−5%, +/−4%, +/−3%, +/−2%, or +/−1%. Likewise, all endpoints of ranges are understood to be individually disclosed, such that, for example, a range of 1:2 to 2:1 is understood to disclose a ratio of both 1:2 and 2:1.

Unless otherwise indicated, all percentages herein are by weight, relative to the weight of the total composition.

The term “surfactants” includes salts of the surfactants even if not explicitly stated.

The term “substantially free” or “essentially free” as used herein means the specific material may be present in small amounts that do not materially affect the basic and novel characteristics of the compositions according to the disclosure. For instance, there may be less than 2% by weight of a specific material added to a composition, based on the total weight of the compositions (provided that an amount of less than 2% by weight does not materially affect the basic and novel characteristics of the compositions according to the disclosure. Similarly, the compositions may include less than 2%, less than 1.5%, less than 1%, less than 0.5%, less than 0.1%, less than 0.05%, or less than 0.01%, or none of the specified material. Furthermore, all components that are positively set forth in the instant disclosure may be negatively excluded from the claims, e.g., a claimed composition may be “free,” “essentially free” (or “substantially free”) of one or more components that are positively set forth in the instant disclosure.

The term “substantially free” or “essentially free” as used herein may also mean that the specific material is not added to the composition but may still be present in a raw material that is included in the composition.

The term “synthetic” means a material that is not of natural origin. The term “natural” means a material of natural origin, such as derived from plants, which also cannot be subsequently chemically or physically modified.

The term “organic” means a material that is produced substantially without or essentially without the use of synthetic materials. The term “substantially without” or “essentially without” as used herein means the specific material may be used in a manufacturing process in small amounts that do not materially affect the basic and novel characteristics of the compositions according to the disclosure. The term “substantially without” or “essentially without” as used herein may also mean that the specific material is not used in a manufacturing process but may still be present in a raw material that is included in the composition.

“Cosmetic composition” encompasses many types of compositions for application to keratin materials such as skin or hair, for example, hair lotions, hair creams, hair gel creams, hair conditioners, hair masques (masks), etc., which can be used either as leave-on or rinse-off treatments or products.

It will be apparent to those skilled in the art that various modifications and variations can be made in the compositions and methods according to the disclosure without departing from the spirit or scope of the disclosure. Thus, it is intended that the disclosure cover such modifications and variations and their equivalents.

### EXAMPLES

The following examples are intended to be non-limiting and explanatory in nature only.

### Example 1

Conditioner formulations comprising the following ingredients were prepared and are disclosed in Table 1 below. Amounts are expressed in wt % of active materials:

TABLE 1
Conditioner Formulations

| | Conditioner | Conditioner | Conditioner |
|---|---|---|---|
| US INCI NAME | Composition A | Composition B | Composition X |
| DISTEAROYLETHYL DIMONIUM | 2.28 | 2.25 | |
| CHLORIDE | | | |
| STEARAMIDOPROPYLDIMETHYLAMINE | | | 1 |
| GUAR | 0.05 | 0.18 | |
| HYDROXYPROPYLTRIMONIUM | | | |
| CHLORIDE | | | |
| HYDROXYPROPYL GUAR | | | 0.05 |
| HYDROXYPROPYLTRIMONIUM | | | |
| CHLORIDE | | | |
| GLYCERYL STEARATE | 1 | 1 | |
| GLYCERYL CAPRYLATE | 0.5 | 0.5 | |
| COCO-CAPRYLATE/CAPRATE | | | 0.5 |
| CETEARYL ALCOHOL | 6.23 | 6.23 | 4.5 |
| C15-19 ALKANE | 1 | 0.5 | |
| VEGETABLE/PLANT OILS - | | | 1.5 |
| ANTHUS ANNUUS (SUNFLOWER) | | | |
| SEED OIL AND/OR GLYCINE SOJA | | | |
| (SOYBEAN) OIL AND/OR OS | | | |
| NUCIFERA (COCONUT) OIL | | | |
| CETYL ESTERS | | | 0.5 |
| ISOPROPYL MYRISTATE | | | 2 |
| BENZYL ALCOHOL | 0.8 | 0.8 | 0.3 |
| GLYCERIN | 4 | 2 | 3 |
| CAPRYLYL GLYCOL | | | 0.3 |
| ADDITIVES - ONE OR MORE OF | <2 | <2 | <2 |
| AMINO ACIDS, TARTARIC ACID, | | | |
| SALICYLIC ACID, SODIUM | | | |
| HYDROXIDE, LACTIC ACID, CITRIC | | | |
| ACID, PLANT/FRUIT EXTRACTS, | | | |
| CARAMEL, FRAGRANCE, | | | |
| PRESERVATIVES | | | |
| WATER | Q.S. 100 | Q.S. 100 | Q.S. 100 |

Shampoo formulations comprising the following ingredients were prepared and are disclosed in Table 2 below. Amounts are expressed in wt % of active materials:

TABLE 2
Shampoo Formulations

| | Shampoo | Shampoo |
|---|---|---|
| | Composi- | Composi- |
| US INCI NAME | tion A | tion B |
| ANIONIC SURFACTANTS: SODIUM | 11 | 20 |
| LAURETH SULFATE AND/OR | | |
| SODIUM LAURYL SULFATE | | |
| AMPHOTERIC SURFACTANTS: | 2.43 | 0.6 |
| COCAMIDOPROPYL BETAINE | | |
| AND/OR COCO-BETAINE | | |
| NONIONIC SURFACTANTS: PPG-5- | — | 3 |
| CETETH-20 AND/OR | | |
| ALKYLPOLYGLUCOSIDE | | |
| GUAR HYDROXYPROPYLTRIMONIUM | 0.1 | |
| CHLORIDE | | |
| GLYCOL STEARATE (and) | — | 1 |
| STEARAMIDE AMP | | |
| CETYL ALCOHOL | — | 2 |
| DICAPRYLYL ETHER | 0.5 | — |
| PENTASODIUM PENTETATE | | 1.1 |
| ADDITIVES - ONE OR MORE OF | <5 | <5 |
| SODIUM HYDROXIDE, SALICYLIC | | |
| ACID, CITRIC ACID, PLANT/FRUIT | | |
| EXTRACTS, SODIUM CHLORIDE, | | |
| HYDROLYZED PROTEIN, FRAGRANCE, | | |
| PRESERVATIVES | | |
| WATER | Q.S. 100 | Q.S. 100 |
| | (70 TO | (70 TO |
| | 85 WT %) | 85 WT. %) |

The formulations were also tested against the following comparative commercial shampoos and conditioners (ingredients listed in order according to label):

| | |
|---|---|
| Comparative Shampoo | Comparative Conditioner |
| Composition Y | Composition Y |

US INCI NAME

| | |
|---|---|
| WATER | WATER |
| AMMONIUM LAURYL SULFATE | BRASSICA ALCOHOL |
| SODIUM COCOAMPHOACETATE | HELIANTHUS ANNUUS HYBRID OIL |
| SODIUM BENZOATE | ESYLATE |
| COCAMIDOPROPYL BETAINE | BUTYROSPERMUM PARKII BUTTER |
| ALOE BARBADENSIS LEAF EXTRACT | COCAMIDOPROPYL BETAINE |
| PHOSPHOLIPIDS | ESCULENTUS ROOT OIL |
| GLYCOLIPIDS | PENTACLETHRA MACROLOBA SEED |
| | OIL |
| GLYCINE SOJA OIL | DICAPRYLYL CARBONATE |
| GLYCINE SOJA STEROLS | DICAPRYLYL ETHER |
| VINEGAR | DECYL GLUCOSIDE |
| GLYCERIN | GLYCERIN |
| LAVANDULA ANGUSTIFOLIA FLOWER | ALOE BARBADENSIS LEAF EXTRACT |
| EXTRACT | |
| ROSMARINUS OFFICINALIS LEAF | GLYCERYL OLEATE |
| EXTRACT | |
| SALVIA OFFICINALIS LEAF EXTRACT | PHOSPHOLIPIDS |
| THYMUS VULGARIS FLOWER/LEAF | GLYCOLIPIDS |
| EXTRACT | |
| YOGURT POWDER | GLYCINE SOJA OIL |
| XANTHAN GUM | GLYCINE SOJ |
| HYDROLYZED WHEAT PROTEIN | ARGININE |
| SODIUM CHLORIDE | TOCOPHEROL |
| PHYTIC ACID | HELIANTHUS ANNUUS (SUNFLOWER) |
| | SEED OIL |
| BENZYL ALCOHOL | BENZYL ALCOHOL |
| POTASSIUM SORBATE | SODIUM BENZOATE |
| LACTIC ACID | BENZOIC ACID |
| BENZOIC ACID | DEHYDROACETIC ACID |
| PARFUM | CITRIC ACID |
| LIMONENE | PARFUM |
| LINALOOL | |

### Example 2

A half head study was conducted involving a panel test of 10 human volunteers with medium length hair, average coarse diameter, with very little or some curls and moderate degree of damage, wherein the performance of conditioner composition A as a conditioner was compared to that of comparative conditioner composition Y (benchmark).

Shampoo composition A was applied to a half head of moderately damaged hair in an amount of 1 g of composition per 2.7 g of hair. The composition was rubbed into the locks for approximately 10-15 seconds. The composition was then rinsed off with water. Conditioner composition A was then applied to the same half head of moderately damaged hair in an amount of 1 g of composition per 2.7 g of hair. The composition was rubbed into the locks for approximately 10-15 seconds. The composition was then rinsed off with water.

Comparative shampoo composition Y was applied to the opposite half head of moderately damaged hair (opposite to the half treated with shampoo composition A and conditioner composition A) in an amount of 1 g of composition per 2.7 g of hair. The composition was rubbed into the locks for approximately 10-15 seconds. The composition was then rinsed off with water. Comparative conditioner composition Y was then applied to the same half head of moderately damaged hair in an amount of 1 g of composition per 2.7 g of hair. The composition was rubbed into the locks for approximately 10-15 seconds. The composition was then rinsed off with water.

It was found that conditioner composition A imparted comparable conditioning properties to the hair locks including suppleness, softness, moisturization, conditioning, detangling, smoothness, discipline, frizz control, and other cosmetic benefits to keratin materials, while maintaining appropriate body/volume/fullness, movement, bounce and style-ability as compared to comparative conditioner composition Y (benchmark). Thus, conditioner composition A according to one disclosed embodiment was able to achieve comparable performance to the comparative composition Y, a commercially formulated composition.

Conditioner composition A was also noted to melt better in the user's hands and be less sticky on the hair than comparative conditioner composition Y.

### Example 3

A half head study was conducted involving a panel test of 10 human volunteers with medium length hair, average coarse diameter, with very little or some curls and moderate degree of damage, wherein the performance of conditioner composition B as a conditioner was compared to that of comparative conditioner composition X (benchmark).

Shampoo composition B was applied to a half head of moderately damaged hair in an amount of 1 g of composition per 2.7 g of hair. The composition was rubbed into the locks for approximately 10-15 seconds. The composition was then rinsed off with water. Conditioner composition B was then applied to the same half head of moderately damaged hair in an amount of 1 g of composition per 2.7 g of hair. The composition was rubbed into the locks for approximately 10-15 seconds. The composition was then rinsed off with water.

Shampoo composition B was applied to the opposite half head of moderately damaged hair (opposite to the half treated with shampoo composition B and conditioner composition B) in an amount of 1 g of composition per 2.7 g of hair. The composition was rubbed into the locks for approximately 10-15 seconds. The composition was then rinsed off with water. Comparative conditioner composition X was then applied to the same half head of moderately damaged hair in an amount of 1 g of composition per 2.7 g of hair. The composition was rubbed into the locks for approximately 10-15 seconds. The composition was then rinsed off with water.

It was found that conditioner composition B imparted comparable conditioning properties to the hair locks including suppleness, softness, moisturization, conditioning, detangling, smoothness, discipline, frizz control, and other cosmetic benefits to keratin materials, while maintaining appropriate body/volume/fullness, movement, bounce and style-ability as compared to composition X (benchmark). Thus, conditioner composition B according to one disclosed embodiment was able to achieve comparable performance as to the comparative composition X, a commercially formulated composition.

1. A composition comprising:
(a) at least one first cationic compound chosen from esterquat compounds;
(b) at least one second cationic compound chosen from:
(i) cationic surfactants other than esterquat compounds, or
(ii) cationic polymers;
(c) at least one hydrocarbon oil;
(d) optionally at least one glyceryl ester; and
(e) at least one fatty alcohol.

2. The composition of claim 1, wherein the at least one first cationic compound is present in an amount ranging from about 0.1% to about 5%, based on the total weight of the composition.

3. The composition of claim 1, wherein the at least one first cationic compound is distearoylethyl dimonium chloride.

4. The composition of claim 1, wherein the at least one second cationic compound is chosen from at least one cationic polymers.

5. The composition of claim 4, wherein the at least one cationic polymer is present in an amount ranging from about 0.05 to about 2%, based on the total weight of the composition.

6. The composition of claim 4, wherein the at least one cationic polymer is guar hydroxypropyltrimonium chloride.

7. The composition of claim 1, wherein the at least one hydrocarbon oil is chosen from alkanes having from 10-30 carbons.

8. The composition of claim 7, wherein the alkane having from 10-30 carbons is present in an amount ranging from 0.1 to about 10%, based on the total weight of the composition.

9. The composition of claim 7, wherein the alkane is a C15-C19 alkane.

10. The composition of claim 1, wherein the at least one fatty alcohol is present in an amount ranging from about 0.5 to about 10%, based on the total weight of the composition.

11. The composition of claim 10, wherein the at least one fatty alcohol is chosen from cetyl alcohol, stearyl alcohol, cetearyl alcohol, behenyl alcohol, lauryl alcohol, myristic or myristyl alcohol, arachidyl alcohol, lignoceryl alcohol, ceryl alcohol, montanyl alcohol, myricylic alcohol, decyl alcohol, undecyl alcohol, or mixtures thereof.

12. (canceled)

13. The composition of claim 1, comprising at least one glyceryl ester, wherein the at least one glyceryl ester is present in an amount ranging from about 0.1 to about 3%, based on the total weight of the composition.

14. The composition of claim 13, wherein the at least one glyceryl ester is chosen from bis-diglyceryl polyacyladipate-2, glyceryl behenate, glyceryl caprate, glyceryl cocoate, glyceryl erucate, glyceryl hydroxystearate, glyceryl isostearate, glyceryl lanolate, glyceryl laurate, glyceryl linoleate, glyceryl myristate, glyceryl oleate, glyceryl palmitate lactate, glyceryl sesquioleate, glyceryl stearate, glyceryl stearate citrate, glyceryl stearate lactate, glyceryl dioleate, glyceryl distearate, glyceryl laurate, or mixtures thereof.

15. (canceled)

16. The composition of claim 1, wherein the composition further comprises a solvent.

17. The composition of claim 16, wherein the solvent comprises water and the composition comprises from about 50% to about 95% water, by weight relative to the weight of the total composition.

18. The composition of claim 1, wherein the weight ratio of the total amount of the first and second cationic compounds to the total amount of the hydrocarbon oil ranges from about 1:10 to about 10:1.

19. (canceled)

20. (canceled)

21. The composition of claim 1, wherein the weight ratio of the total amount of the first and second cationic compounds to total amount of the glyceryl esters ranges from about 1:10 to about 10:1.

22. (canceled)

23. (canceled)

24. The composition of claim 1, wherein composition is substantially free of synthetic gums, silicones, and/or synthetic polymers.

25. (canceled)

26. A method for treating hair, said method comprising:
(1) applying to the hair a composition comprising:
(a) at least one first cationic compound chosen from esterquat compounds;
(b) at least one second cationic compound chosen from:
(i) cationic surfactants other than esterquat compounds; or
(ii) cationic polymers;
(c) at least one hydrocarbon oil;
(d) optionally at least one glyceryl ester; and
(e) at least one fatty alcohol, and
(2) rinsing the hair.

27. The method of claim 26, wherein step (1) is conducted after treating the hair with a shampoo.
